(19) United States
(12) Patent Application Publication
(10) Pub. No.: US 2022/0265877 A1
(43) Pub. Date: Aug. 25, 2022

(54) Improvements in and Relating to Sterilisation of Fluid-Guiding Elements for Bioprocessing Applications

(71) Applicant: Cytiva Sweden AB, Uppsala (SE)

(72) Inventor: Hanno Ehring, Uppsala (SE)

(21) Appl. No.: 17/620,617

(22) PCT Filed: Jun. 17, 2020
(86) PCT No.: PCT/EP2020/066747

§ 371 Date: Dec. 17, 2021

(30) Foreign Application Priority Data
Jun. 28, 2019 (GB) 1909315.2

(51) Int. Cl. A61L 2/10; A61L 2/26

(52) U.S. Cl. CPC A61L 2/10; A61L 2/26; A61L 2202/11

(57) ABSTRACT

Disclosed is an ultra-violet (UV) light sterilisable fluid-guiding element (100, 200, 300, 400, 500) configurable to form a part of a normally closed bioprocessing fluid system, at least a portion of the element being formed from a material which is transmissive to UV light, said at least one portion of the element including one or more surfaces (134) configured to contact and guide fluids within the closed system. The element further includes at least one UV light emitting diode (LED) (154) mounted in, on, or adjacent the at least one portion and has sufficient light output to sterilise at least the one or more surfaces (134).

### FIELD OF THE INVENTION

The present invention relates generally to the sterilisation of fluid-guiding elements by ultra-violet (UV) light, and more particularly to the use of such fluid-guiding elements for bioprocessing applications.

### BACKGROUND OF THE INVENTION

Herein, ‘sterile’ and similar terms such as ‘aseptic’ are intended to mean a bio-burden reduced to a level sufficient for the needs of the intended purpose of the elements, and so for practical purposes, herein, sterilisation of an element means to reduce the bio-burden enough for the element to perform its function without introducing undue bio-burden.

Generally, many closed fluid systems required sterile conditions in order to perform successfully. For example, bioprocessing, medical equipment, food processing, brewing, and water treatment are some examples where closed sterile systems are employed which are pre-sterilised and kept sealed to maintain sterility.

Prior to use, such systems can be cleaned with chemicals such as sodium hydroxide, ethylene oxide gas with steam or with radiation, such as gamma radiation, or UV light. Of course, not all of those methods are acceptable for all closed fluid systems.

However, in many instances access to the otherwise closed system is needed, for example to extract or part-extract finished fluids or products, or for sampling, or to introduce fluids such as reagents or ingredients, or to monitor the fluids using probes or sensors. In those instances, there is always the risk of introducing contaminants, such as bacteria, fungi, viruses, enzymes and like simple-form life into the system, herein collectively termed ‘microorganisms’.

So, in-process sterilisation and cleaning are required in many instances, even after initial sterilisation of the empty system. One particularly problematic system is cell culture in a bioreactor, where a normally closed system is kept in a condition which promotes microorganism growth, and where many invasive procedures are conducted during the cell culture process. For example, the initial introduction of seed cells, the introduction of oxygen and cell nutriments, the sampling of cells, and the harvesting of the cells or cell products such as antibodies or other proteins.

To address potential contamination, various couplings and techniques have been proposed which purport to maintain sterility when the system is opened, but absolute sterility can never be assured with such mechanical couplings, even in so-called clean-room conditions, where there is human activity.

Commercially available sterile couplings have been proposed, for example as proposed in U.S. Pat. No. 6,679,529, WO 2009/002468 and WO 2013/147688, and sold under the brand name ReadyMate® which provide one of the best methods of ensuring sterility when connecting two otherwise sterile fluid systems together, whereby a pair of connections are brought together each being covered by a film, and once the connections are brought together the films are removed leaving the connections mutually connected but never exposed to their environment. There are many other mechanical concepts using male and female parts which are exposed after mating. Their shortcomings are that careful manual manipulation is needed to make a hopefully sterile connection.

Additionally, one major issue with prior art system designs when used for bioprocessing applications relates to the problem of controlling microorganisms that can be trapped in joints or stagnant zones of, for example, bioreactors. Such systems can be hard to keep clean whilst in use and are required to remain sterile when in use. Any stagnant zones are thus highly undesirable since they may trap biological material that may degrade and subsequently release pathogens or other undesirable materials into a processing batch.

Any such contamination may thus result in a whole batch of biopharmaceutical products needing to be thrown away.

### SUMMARY

The inventor of the present concept has realised that, even with the best sterile connections, there will always be a risk of microorganism contamination and so a different approach is required, and conceived that a better method for sterilisation before, during or after coupling two fluid systems was required. The inventor proposes the use of UV light transmissive fluid connections or other fluid-guiding elements which form part of a closed fluid system, such as tube couplers, fluid transfer ports, valves, fluid sampling interfaces, removable sensor ports, bungs, and anywhere where it is possible for microorganisms to cross from the outside environment into a normally closed fluid system, or in areas where conventional pre-sterilising methods cannot reach, such as joints which are too narrow to allow cleaning fluids to pass, or stagnant areas (so-called dead legs). When exposed to UV light those fluid-guiding elements can transmit UV light to their fluid contacting surfaces, before and/or after they are connected to the closed system, in order to sterilise the fluid contacting surfaces and adjacent regions.

The ultraviolet (UV) light spectrum lies between 200 and 400 nanometres. The so-called UV-C part of this (wavelength of around 254 nanometres) is particularly suitable for sterilisation because it has been found to be capable of destroying microorganisms, or at least damages their DNA which prevents, for example viruses or spores from then infecting cells. UV light of about 255-265, e.g. about 260 nanometres emitted from commercially available LEDs is particularly suitable because it is UV=C radiation and little or no heat is produced, meaning that such LEDs can be placed close to sensitive parts or liquids with no risk of damaging them by means of heat. The LED's size also renders it suitable for placing in on or adjacent the fluid-guiding elements, to provide local sterilising UV light.

It should be understood that the summary above is provided to introduce, in simplified form, a selection of concepts that are further described in the detailed description below. It is not meant to identify key or essential features of the claimed subject matter, the scope of which is defined uniquely by the claims that follow the detailed description. Furthermore, the claimed subject matter is not limited to implementations that solve any disadvantages noted above or in any part of this specification. The above advantages and other advantages and features of the present description will be readily apparent from the following Detailed Description when taken alone or in combination with the accompanying drawings. The invention extends to any combination of features disclosed herein, whether or not such a combination is mentioned explicitly herein. Further, where two or more features are mentioned in combination, for example in the same paragraph, it is intended that such features may be claimed separately without extending the scope of the invention. Features from different embodiments described below may be brought together in any claim.

In its broadest form, the invention provides a fluid-guiding element according to claim 1 having preferred features defined by claims dependent on claim 1.

The invention provides also a method as defined by claim 12.

### BRIEF DESCRIPTION OF THE DRAWINGS

The invention can be put into effect in numerous ways, illustrative non-limiting embodiments of which are described below with reference to the drawings, wherein:

FIG. 1 shows a first embodiment of a fluid-guiding element in the form of a tube coupling;

FIG. 2 shows a second embodiment of a fluid-guiding element in the form of a modified tube coupling;

FIGS. 3a, 3b and 3c show different operational positions of a third embodiment of a fluid-guiding element; and

FIGS. 4 and 5 show further embodiments of the fluid-guiding element.

### DETAILED DESCRIPTION

The invention, together with its objects and the advantages thereof, may be understood better by reference to the following description taken in conjunction with the accompanying drawings, in which like reference numerals identify like elements in the Figures.

Referring to FIG. 1 there is shown a fluid-guiding element in the form of a fluid coupling 100. The coupling 100 includes a body 130, which has a barb 132 to accept a plastics tube 120 which is clamped to the barb by means of a clamp 122, and a mating face 110. The coupling 100 is intended to fit to a complementary coupling (not shown) at its mating face 110. In use the inner surface 134 of body 130 and the mating face 110 come into contact with fluids. In use the mating face 110 is offered up to the complementary face and once their respective ring seals 112 are in contact, their respective sealing films 114 are pulled out together from the gap between the faces, provided by ring seals. The complementary couplings can be held together by means of a ring clamp (not shown) which holds their respective outer flanges 136 together. That system allows good assurance of maintaining sterility of pre-sterilised couplings, but once broken the coupling becomes contaminated, potentially contaminating the fluid system.

In this embodiment a sterilising UV LED circuit 150 is provided, in an extension 140 of the housing 130 which includes a push to make switch 152, a UV LED 154, a battery 156, and a current limiting resistor 158. The LED provides, on demand, UV sterilising light, which can reflect internally throughout the housing 130 across the area shown as hatched lines, even into any voids or tight joints, for example the void 138 at the external surface of the barb, which could harbour microorganisms.

In use the fluid contacting surfaces 134 and 110 of the coupling can be sterilised prior to coupling with another similar coupling, and/or after making that coupling. If the joined tubes 120 or equivalent parts are made transparent also, then UV light will propagate by internal reflection also along the tubes, at least for a little distance, so they will be partially sterilised also. In a refinement, an outer surface 144 of the body 130 has UV light reflective properties, for example a polished and metal plated surface or an internally mirrored surface, to better reflect light back toward the fluid contacting surfaces 134.

In various alternative embodiments, UV LED circuitry may be provided that is remotely controllable, contactlessly powered and/or transistor switch operated. Such UV LED circuitry may be provided for multiple bioprocessing system components and centrally operated, e.g. by a remote computer system. This can thereby enable the provision of an automated bioprocessing system which can be operated in a co-ordinated manner to improve sterilisation efficiency.

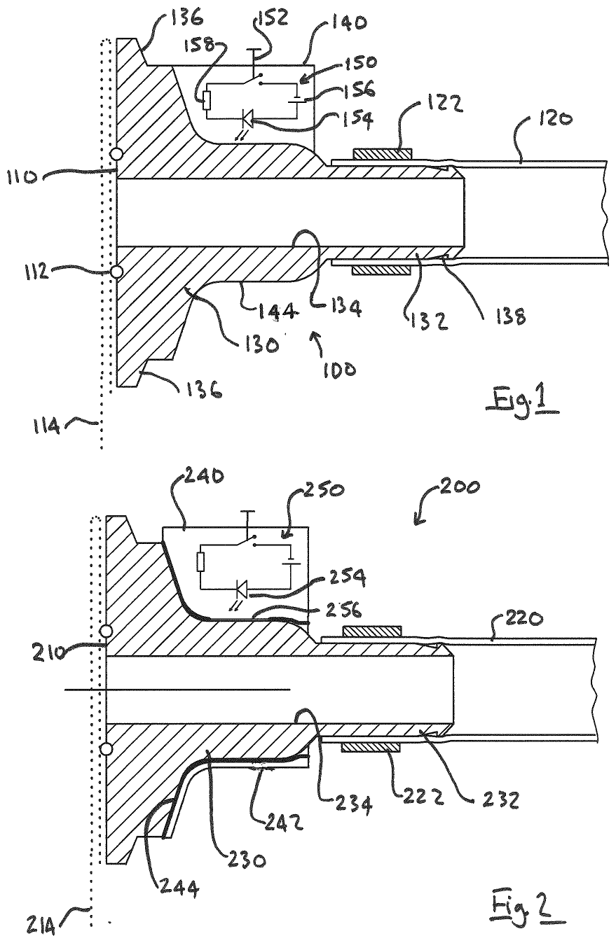

FIG. 2 shows another fluid-guiding element 200 in the form of a fluid coupling similar to the coupling 100, where similar parts have the same last two digits. In this embodiment, fluid contacting surfaces 234 and 210 are again sterilised in use with light internally reflected from a UV LED circuit 250, as explained above. In this case the extension 240 which houses the LED circuit 250 also includes a shroud 242 which fits snuggly over the body 230 and has a highly reflective surface 244, such as polished metal, or an electroplated metallised finish, on surfaces adjacent the body 230, except for an area 256 next to the LED 254 to allow light into the transparent body 230. This arrangement is more efficient than the embodiment of FIG. 1 because more of the LED light is recycled by the reflective surface and can be retrofitted to existing couplings which do not have provision for an LED circuit. As an alternative, the body 230 could have a highly reflective (with a window equivalent to window 256, for example a mirrored outer surface, in place of the reflective shroud 242, in which case the shroud 242 would function merely as a removable mount for the UV circuit 250, including its adjacent LED light source 254.

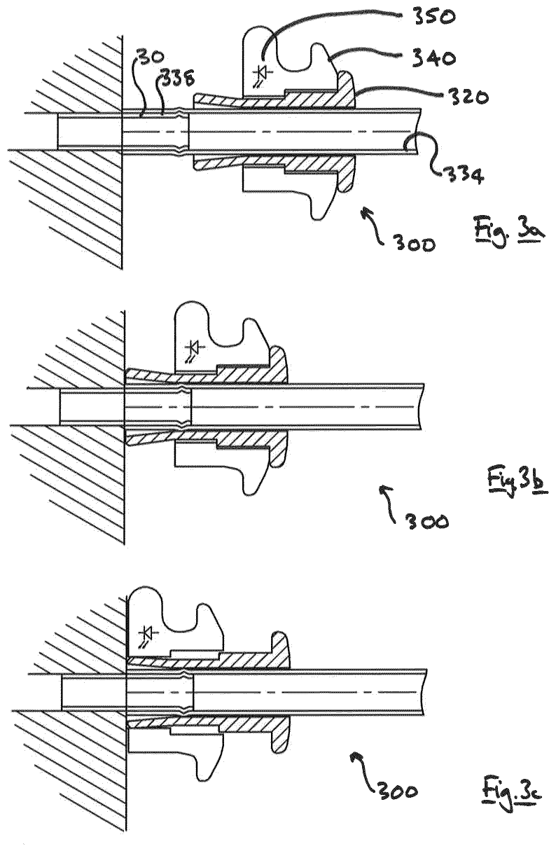

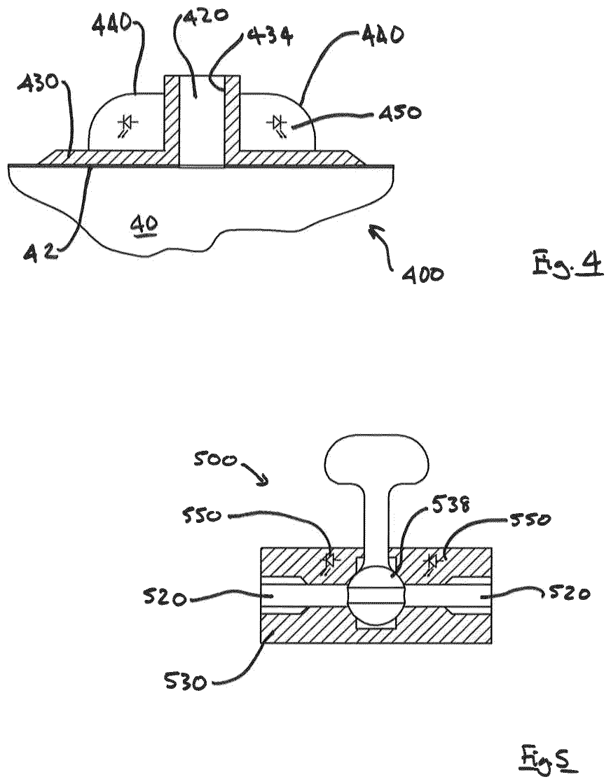

FIG. 3a shows anther fluid-guiding element 300, on the form of a fluid tube 320 and a two-piece tube clamp 330/340. Here it is intended that the outer tube clamp 340 houses an LED circuit 350, for providing sterilising light to an inner tube clamp 330. Provided the tube 320 is transparent also to UV light, the propagation of UV light through the inner tube clamp 330 will sterilise by means of internal reflection the inner surface 334 of the tube 320, and the tube joint 338 between the tube 320 and its spigot 30 which fits inside the tube 320.

With reference additionally to FIGS. 3b and 3c, it can be seen that the inner clamp 330 is, in use, slid axially over the spigot 30, and then the outer clamp 340 is slid axially over the inner clamp 330, to squeeze the inner clamp 330 around the tube 320 to hold it in place on the spigot 30. During that clamping process the UV LED can be illuminated to provide a period of time and some relative movement of the LED to provide complete sterilisation of the coupling 300.

FIG. 4 shows a fluid-guiding element in the form of a port 400, suitable for accessing a bioprocessing container 40, for example for supplying fluids, for withdrawing fluids, for sampling or for inserting a probe or sensor into the container. When not in use the port is sealed, for example using a screw cap (not shown). The port 400 includes a body 430 which is transparent to UV light and has a fluid access aperture 420, and a body extension 440 which houses a UV LED circuit 450. The body is transparent and allows UV light to propagate to the fluid contacting surface 434 of the aperture 420, before, during and/or after use of the port. In this instance, the port is likely to be used next to cells or other bio-materials, and so the wall 42 of the container 40 is made from non-UV Led transmitting plastics to prevent damage to the container's contents from stray UV light.

FIG. 5 shows a fluid-guiding element in the form of a ball valve 500 including a valve body 530, which houses a UV LED circuit 550 containing multiple LEDs. The body 550 is transparent to UV light and includes fluid passages 520, as well as rotary valve mechanism 538 each of which can be sterilised by allowing light to propagate by internal reflection around the body to the passages 520 and to the valve joints between the body 530 and the mechanism 538. Thereby the various passages of the valve and any joints or dead legs can be sterilised before and after use of the valve. Alternative valve arrangements could also be sterilised in this way.

It will be apparent that other arrangements similar to those mentioned above could be used such that fluid-guiding element can be sterilised by UV light for its subsequent use in a generally closed fluid system.

Whilst it is possible for the sterilisation to be performed with weakly UV transmissive materials, i.e. those materials that do not transmit light particularly well, or absorb UV light, provided at least 5% UV light is transmitted per mm (5%/mm) thickness of the element, then effective sterilising can be assured.

Materials useful for transmitting UV light are numerous, for example glass, for example quartz glass is suitable. However, for disposable products which are typically used in bioprocess and medical applications, a plastic would be more economical. In this regard, it is also typical to gamma pre-sterilise the plastics components before use, which for some plastics reduces their UV light transmission properties. Suitable plastics are: polypropylene (PP), although UV degradation over time is an issue; polymethyl methacrylate (PMMA), which is not particularly UV transmissive to light at wavelengths shorter than 250 nm, but at around 260 nm wavelength it's transmissive properties are acceptable, i.e. about 50%/mm; polydimethylsiloxane (PDMS), although it is not easy to injection mould; Polyimide (PI) for example fluorinated PI, which is stable and transmits all UV light when made colourless.

Where less transmissive materials are employed, or where faster sterilisation is required, then more than one LED can be used, for example a ring of LEDs can be used in any of the embodiment described above.

Although exemplary embodiments have been described and illustrated, it will be apparent to the skilled addressee that additions, omissions and modifications are possible to those embodiments without departing from the scope of the invention claimed. For example the circuits 150,250, 350,450 and 550, are intended, for convenience, to have a local power supply, for example from a battery. That arrangement suits disposable type fluid-guiding elements such as disposable fluid couplings, but where reusable elements are required, a remote power source could be used, for example a plug-in dc supply. Only the embodiment of FIG. 1 is described as optionally including a back-reflective external surface, although the other embodiments could also include such a surface.

1. An ultra-violet light sterilisable fluid-guiding element for bioprocessing applications which is configurable to form a part of a normally closed fluid system, at least a portion of the element being formed from a material which is transmissive to UV light, said at least one portion of the element including one or more surfaces configured to contact and guide fluids within the closed system in use, the element further including at least one UV light emitting diode mounted in, on, or adjacent the at least one portion and having sufficient light output to sterilise at least the one or more surfaces.

2. The fluid-guiding element of claim 1, wherein the LED emits light of a wavelength of about 100 to about 280 nanometres, for example 260 nanometres.

3. The fluid-guiding element of claim 1 or 2, wherein the material transmits 5% or more of the UV light per mm light propagation distance, preferably more than about 10% per mm, more preferably more than about 20% per mm.

4. The fluid-guiding element of claim 1, wherein the material is glass, for example a quartz glass, or is a polypropylene or a polymethyl methacrylate, or a polydimethylsiloxane, or a polyimide for example fluorinated PI, or a combination of said materials.

5. The fluid-guiding element of claim 1, wherein said fluid-guiding element is one or more of: of a fluid connector, a tube coupler, a removeable fluid transfer port, a valve, a fluid sampling interface, a removable sensor port, a bung, or an element at a location where it is possible for microorganisms to cross from an outside environment into the normally closed fluid system, or an element at a location where conventional pre-sterilising is ineffective.

6. The fluid-guiding element of claim 1, wherein the element includes a body having said one or more surfaces configured to contact and guide fluids and the body supports a circuit for operating the at least one LED.

7. The fluid-guiding element of claim 6, wherein the circuit is provided within an extension of the body.

8. The fluid-guiding element of claim 6, wherein the circuit comprises a push to make switch, a UV LED, a battery, and a current limiting resistor.

9. The fluid-guiding element of claim 6, wherein an external surface of the body includes a highly reflective region for back-reflecting UV light.

10. The fluid-guiding element of claim 6, further including a shroud for support said circuit, the shroud optionally having a reflective surface adjacent the body for back-reflecting UV light.

11. The fluid-guiding element of any preceding claim comprising a body or a housing that is configured to reflect UV light into at least one void which could harbour microorganisms.

12. A method for maintaining the sterility of a generally closed fluid system for bioprocessing applications, the method including the steps of:
a) opening the fluid system at a fluid-guiding element which has at least a portion of the element making contact with the fluid;
b) closing the fluid system at the element; and
c) sterilising the portion of the element by means of UV light transmitted to the portion making fluid contact by propagation through the element, before and/or during and/or after closing.

13. The method of claim 12 wherein said UV light is light from an LED having an output wavelength of about 100 to about 280 nanometres, for example 260 nanometres.
